(19) United States
(12) Patent Application Publication
(10) Pub. No.: US 2006/0210568 A1
(43) Pub. Date: Sep. 21, 2006

(54) Methods and compositions for cancer treatment

(76) Inventor: Ginette Serrero, Ellicott City, MD (US)

Correspondence Address: CROWELL & MORING LLP;INTELLECTUAL PROPERTY GROUP, P.O. BOX 14300, WASHINGTON, DC, 20044-4300, US

(21) Appl. No.: 10/548,353

(22) PCT Filed: Mar. 8, 2004
(86) PCT No.: PCT/US04/06814

§ 371 Date: Sep. 7, 2005

(30) Foreign Application Priority Data
Mar. 7, 2003 (US) 60452525

(51) Int. Cl. A61K 39/395; A61K 38/17; A61K 31/426; A61K 31/203; A61K 31/202; A61K 31/19; A61K 31/192; A61K 31/405

(52) U.S. Cl. 424/155.1; 514/12; 514/559; 514/558; 514/560; 514/420; 514/570; 514/369

(57) ABSTRACT

Methods and compositions for breast cancer treatment and prevention by inducing overexpression of adipose differentiation related protein (ADRP) in the cancer cells. Over expression of ADRP causes breast cancer cells to re-differentiate, whereby cancer cell proliferation and tumorigenesis is inhibited and cancer cells are induced to undergo apoptosis. Also disclosed are pharmaceutical compositions comprising polynucleotides encoding ADRP, ADRP polypeptides or analogs or mimetics thereof, and methods for screening for ADRP agonists.

### FIELD OF THE INVENTION

This invention relates to methods and compositions for breast cancer treatment and prevention by stimulating breast cancer cells to re-differentiate, whereby cancer cell proliferation is inhibited and cancer cells are induced to undergo differentiation or apoptosis. Specifically, this invention relates to methods and compositions that induce overexpression of adipose differentiation related protein (ADRP).

### BACKGROUND OF THE INVENTION

Among women, breast cancer is the most commonly diagnosed cancer after non-melanoma skin cancer, and is the second leading cause of cancer deaths (Menck et al., 1997, CA Cancer J. Clin. 47:161-170). In 2002, an estimated 205,000 new cases were diagnosed and 40,000 deaths from breast cancer occurred in the United States. (American Cancer Society: Cancer Facts and Figures 2002. Atlanta, Ga.). Breast cancer also occurs in men, although much more rarely than in women (Kumar et al., 2000, Endocrine Related Can. 7:257-269). There are different types of treatment currently available to patients with breast cancer, including surgery, radiation therapy, chemotherapy and hormone therapy. New treatment methods, however, are desired that overcome the drawbacks in many of the currently available methods. Several new treatments are being tested in clinical trials, such as sentinel lymph node biopsy followed by surgery, and high-dose chemotherapy with bone marrow transplantation and peripheral blood stem cell transplantation.

Adipose Differentiation related protein (ADRP) is a 50-kDa protein expressed at very high level in adipose tissue in mature differentiated adipocytes, but at very low level in undifferentiated adipocyte precursors. The presence of ADRP was generally considered an indication that the cells are differentiated. In other words, ADRP has been considered a “differentiation marker.”

ADRP is generally localized on the surface of lipid droplets, and plays a structural role in neutral lipid deposition and mobilization by participating in the formation of the membrane surrounding the lipid droplet. ADRP was found on the surface of lipid droplets in many cells, including adipocytes, fibroblasts, and human liver cells HepG2, regardless whether the lipid droplets contain neutral lipids (triglycerides), such as in adipocytes, or steroids, such as in adrenal cells (Heid et al., 2000). In cos-7 cells, overexpressed ADRP was found localized to the periphery (Gao and Serrero, 1999, J. Biol. Chem. 274:16825-16830)

ADRP is a fatty acid binding protein and stimulates uptake of long chain fatty acids in cells. This was shown by two different methods. In the first approach (Gao and Serrero, 1999, J. Biol. Chem. 274:16825-16830), ADRP cDNA was inserted in a mammalian expression vector system called pcDNA3 that contains a cytomegalovirus (CMV) promoter for constitutive expression of gene product. The ADRP pcDNA3 construct was transfected into Cos-7 cells by the DEAE dextran method, and uptake of radiolabeled fatty acids was determined in ADRP transfected cells and in empty vector control Cos-7 cells. Using an anti-ADRP polyclonal antibody raised in the laboratory, ADRP expression was localized to the cell surface and was shown to be associated with an increase in the uptake of long chain fatty acids into the cells, while no change of uptake of short chain fatty acids was observed. The second approach (Serrero et al., 2000, Biochim. Biophys. Acta. 1488: 245-254) was to express recombinant mammalian ADRP in bacteria and purify the protein under experimental conditions that maintained its biological activity. It was shown that the purified recombinant ADRP was biologically active and was able to bind fluorescent fatty acids. In addition to binding fatty acids, ADRP could also bind sterols and possibly cholesterol, making it a lipid-binding protein with a large spectrum of lipid classes (Frolov et al., 2000, J. Biol. Chem. 275: 12769-80).

ADRP cDNA was originally cloned by the present inventor from a mouse adipocyte cDNA library with differential screening for cDNA highly expressed in differentiated adipocytes but at very low level in undifferentiated adipocyte precursors. (Jiang et al., 1992, Cell Growth & Differentiation 3:21-30; Jiang and Serrero, 1992, Proc. Natl. Acad. Sci. USA 89:7856-7860, and U.S. Pat. No. 5,268,295). The mouse ADRP gene has been sequenced and located on chromosome 4 at locus adfp (Eisinger and Serrero, 1993, Genomics 16:638-644). Human homologue of ADRP, also termed adipophilin has been found to be expressed in a variety of tissue and cell lines (Brasaemle et al., 1997, J. Lipid Res. 38:2249-2263; Heid et al., 1998, Cell & Tissue Res 294:309-321). Expression control of ADRP has been studied in the present inventor's laboratory and ADRP expression in adipocytes has been shown to be stimulated by ibuprofen and indomethacin (Ye and Serrero, 1998, Biochem. J. 330:803-809). In addition, long chain fatty acids (either metabolizable or non-metabolizable), but not short chain fatty acids, also stimulate ADRP expression in these cells (Gao et al., 2000, J. Cell Physiol. 182:297-302).

As a normal cell matures and differentiates, it becomes committed to its lineage and its proliferative capacity decreases and may eventually cease. Cancer cells, in contrast, remain in an undifferentiated or de-differentiated state and proliferate uncontrollably, because the genes or other mechanisms involved in controlling the cell cycle and division are altered or blocked. For example, it has been shown that transfection of Her2/Neu (erbB2) will lead the cells to de-differentiate, which in turn stimulates growth of human breast cancer cells (Lazar et al., 2000, Int. J. Cancer 85:578-83). This process of dedifferentiation takes the cells out of G1 phase (where they are when differentiated) and makes them reenter the cell cycle where they proliferate and become cancerous.

Two approaches are generally used for cancer treatment. The first and most widely used approach is to directly inhibit cancer cell proliferation, including killing cancer cells directly. The other approach is to stimulate cancer cells to differentiate, thereby stopping cell proliferation. The notion of treating human malignancies by forcing cancer cells to complete terminal differentiation was first suggested by Pierce (1961, Can. Cancer Conf. 4:119-137). Once the cells are stimulated to differentiate, they will exit the cell cycle, stop proliferating and enter the G1 phase. This approach will also stimulate apoptosis of the cells that failed to differentiate thereby eliminating the proliferating cells. This second method has also the advantage of being applicable to chemoprevention of cancer, because a differentiation selection pressure is maintained to prevent cells from exiting G1 phase and from proliferating. For example, when treated with retinoic acid, HL-60 promyelocytic leukemia will differentiate into polymorphonuclear leukocytes (Pisano et al., 2002, Blood 100:3719-3730). In addition, it was shown that human breast cancer cells can be induced to undergo terminal differentiation by the activation of PPARγ, a nuclear receptor that is expressed in insulin responsive tissues but is also expressed at lower levels in many other tissues (Mueller et al., 1998, Molecular Cell 1:465-470).

As discussed above, ADRP was generally considered as an indicator that the cells are differentiated. For example, U.S. Pat. No. 5,268,295 specifically states that ADRP is expressed in high quantities in adipogenic cells after cell differentiation. There was no suggestion that ADRP induces or stimulates cell differentiation even in adipocytes, and there was no teaching or suggestion in the prior art suggesting that there might be a link between ADRP and breast cancer.

### SUMMARY OF THE INVENTION

This invention is based on the surprising discovery that ADRP is a differentiation inducer, and its over-expression stimulates breast cancer cells to re-differentiate whereby cancer cell proliferation is inhibited and the cancer cells are induced to undergo terminal differentiation or apoptosis. The present invention shows that over-expression of ADRP sensitizes cancer cells to apoptosis stimuli, thereby making them more sensitive to cytotoxic killing. Accordingly, inducing ADRP over-expression can be used to increase killing of drug resistant breast cancer cells.

This invention generally provides a method for treating or preventing breast cancer in a patient, the method comprising inducing ADRP gene over-expression in a breast cell of the patient. Preferably, the breast cell is a cancer cell. Specifically, the method comprises increasing ADRP level or inducing endogenous ADRP gene expression of said breast cell.

According to one embodiment, the endogenous ADRP gene is induced to over-express by administering an effective amount of a compound selected from the group consisting of a 1-PPARγ ligand, 9-cis retinoic acid, a long chain fatty acid, estradiol, and a 4-Cyclooxygenase inhibitor. Preferably, the PPARγ ligand is ciglitazone or thialozidinedione (TZD). The long chain fatty acid is preferably a non-metabolizable long chain fatty acid, such as bromopalmitate, and the 4-Cyclooxygenase inhibitor is preferably indomethacin or ibuprofen.

According to another embodiment of the present invention, the ADRP level of the cell is increased by delivering ADRP protein to the cell. Preferably, the ADRP protein is delivered to said cell via a breast cell-specific antibody.

According to yet another embodiment, the method comprising introducing an exogenous polynucleotide encoding ADRP into a breast cell and expressing said exogenous ADRP gene. Preferably, the exogenous polynucleotide encoding ADRP is operably linked to a promoter. Preferable promoter is a mammary tissue-specific promoter, such as an ADRP promoter, whey acidic protein promoter, beta casein promoter, Lactalbumin promoter and Beta-lactoglobulin promoter. Suitable promoter for the method of the invention may also be a constitutive or an inducible promoter.

The present invention further provides for a method for screening for an agent for treating or preventing breast cancer, said method comprising 1) providing a cell which comprises an ADRP promoter operably linked to a reporter gene, 2) admixing a test agent to the cell, 3) determining expression level or activity of said reporter gene in the presence of the test agent, 4) determining expressing level or activity of said reporter gene in the absence of the test agent, and 5) selecting test agents that increases ADRP promoter activity. Preferably, the cells are Cos-7 cells, CHO cells, human 293 cells, Hela cells or mammary epithelial cells, and the reporter gene is a luciferase gene.

In a further embodiment, the invention provides for a method for screening for an agent for treating or preventing breast cancer, the method comprising: 1) providing a cell which expresses ADRP protein; 2) admixing a test agent to the cell, 3) measuring an activity of ADRP in the presence of the test agent, 4) measuring an activity of ADRP in the absence of the test agent, and 5) selecting test agents that increase the ADRP activity. Suitable ADRP activities for such screening method include fatty acid uptake, fatty acid binding, and induction or increase of a differentiation marker of mammary epithelial cells. Preferably, the differentiation marker is beta casein expression or accumulation of lipid droplets. A particularly preferable ADRP activity is lipid uptake of ADRP-expressing cells. Another preferable ADRP activity is stimulation of breast cancer cell differentiation, inhibition of proliferation of human breast cancer cells, or stimulation of breast cancer cell apoptosis.

The present invention further provides for a pharmaceutical composition comprising an effective amount of an ADRP polypeptide or polynucleotide or an ADRP agonist, for breast cancer treatment, and a pharmaceutically acceptable excipient.

### BRIEF DESCRIPTION OF THE DRAWINGS

FIG. 1 shows growth curves of MCF7 EV (which do not over-express ADRP) cells and MCF7-C4 cells (which over express ADRP) in serum containing media with and without estradiol.

FIG. 2 shows growth rates of MCF7 EV cells, and MCF7-C4 cells, as measured by thymidine incorporation in various media supplemented with various growth factors known to stimulate DNA synthesis of mammary epithelial MCF-7 cells.

FIG. 3 shows that over-expression of ADRP in MCF-7 cells (MCF-7 C1 or MCF7 C4 cells) lead to stimulation of casein expression (a marker of mammary epithelial cells differentiation) (FIG. 3A, two center lanes). Ciglitazone, an agent that stimulates casein expression FIG. 3A, last lane), also stimulates ADRP expression in MCF-EV cells (FIG. 3B).

FIG. 4A shows the comparison between Oil Red O staining of COS-7 cells transfected with ADRP-pcDNA3 and empty pcDNA3 vectors. FIG. 4B provides a quantitation of the lipid droplets accumulated in ADRP-overexpressing cells when compared to MCF-7 EV cells. There is about a 5-fold increase in the number of lipid droplets accumulated in ADRP overexpressing cells than in EV-MCF-7 cells that express undetectable (to low level) of ADRP.

FIG. 5 shows that the ADRP-overexpressing cells (ADRP-MCF) express much lower level of bcl-2 than the MCF-7 EV cells, making the ADRP-overexpressing cells more susceptible to undergo apoptosis.

FIG. 6 shows that the ADRP-overexpressing cells show a dramatic inhibition of anchorage independent growth when compared to MCF-7 EV cells indicating that overexpression of ADRP results in an inhibition of the breast cancer cells tumorigenesis.

FIG. 7 shows that estradiol stimulates ADRP expression in MCF-7 cells.

FIG. 8 shows that inhibition of ADRP expression in the human breast cancer cell MCF-7 blocks its differentiation.

FIG. 9 shows that inhibition of ADRP expression by hADRP siRNA transfection does not block the ability of ciglitazone to stimulate PPAR-γ activity.

### DETAILED DESCRIPTION OF PREFERRED EMBODIMENTS

The present invention provides methods and compositions for treating breast cancer whereby cancer cell proliferation is inhibited and/or the cancer cells are induced to undergo apoptosis. In one embodiment, breast cancer cells are induced or stimulated to enter the differentiation process by one or more differentiation inducers.

As used in the present invention, a “differentiation inducer” is a factor, which may be a gene product, which when introduced into or otherwise applied to a cell will trigger the differentiation process of the cells and allow them to acquire a functional phenotype. Suitable differentiation inducer may be a macromolecule such as a polynucleotide, a polypeptide, or it may be a small molecule. Based on the surprising discovery that ADRP (over)-expression induces breast cancer cells to differentiate, a preferred differentiation inducer suitable for the present invention is a polynucleotide that encodes a polypeptide having an ADRP activity hereinafter an “ADRP polynucleotide” or an “ADRP gene”). This polynucleotide may be introduced into a cell, e.g. a breast cancer cell, and expressed, to induce the cell to differentiate or re-differentiate.

In a preferred embodiment, the ADRP-encoding polynucleotide is under the control of at least one suitable regulatory element and is expressed or over-expressed in a target cell, preferably a cancer cell. By “expressed or over-expressed” it is meant that the ADRP-encoding polynucleotide is expressed in the cell to produce a level of ADRP that is at least 2-fold, preferably at least 5 fold, or at least 10 fold, above the endogenous ADRP level in the target cell and preferably sufficient to induce the target cell to enter into a differentiation or re-differentiation process.

In a preferred embodiment, the method for treating or preventing breast cancer in a patient according to the present invention comprises inducing ADRP gene over-expression in cells of the breast of the patient. Breast epithelial cells most susceptible to develop into tumors are ductal cells and lobular cells since 90% of breast cancer are ductal carcinoma and the rest is lobular carcinomas. Accordingly, breast ductal cells and lobular cells are preferably targeted for increased ADRP expression. Breast alveolar cells and stromal cells may also be targeted, at least for cancer chemoprevention.

The ADRP gene to be over expressed could be an endogenous gene of the cell. It is well known that ADRP gene exists in all breast cancer cells and its expression level is generally low. The expression level of the endogenous gene may be increased by, e.g., administering an effective amount of a compound known to stimulate ADRP expression. These compounds include but are not limited to a 1-PPAR-γ ligand such as ciglitazone or thialozidinedione (TZD), 9-cis retinoic acid, a long chain fatty acid, estradiol, and a 4-cyclooxygenase inhibitor such as indomethacin or ibuprofen. A suitable long chain fatty acid may be a metabolizable or a non-metabolizable long chain fatty acid, such as bromopalmitate. Preferably, of the level of ADRP may be increased by at least about two fold of the endogenous level, and may be as high as possible. An increase over 10-fold is observed by treatment with ciglitazone and about 2-3 fold with estradiol. This level of increase also depends on the length of treatment.

In another embodiment, the method of the present invention increases the ADRP level of a target cell by delivering the ADRP protein or an analog, fragment or mimetic thereof that has a requisite biological activity of ADRP to the cell. This suitable ADRP protein or analog or fragment may be directly administered to the patient, preferably in situ if necessary because breast cancer is localized.

Human and mouse ADRP genes have been cloned (Jiang et al., 1992, Cell Growth & Differentiation 3:21-30; Jiang and Serrero, 1992, Proc. Natl. Acad. Sci. USA 89:7856-7860, and U.S. Pat. No. 5,268,295). Therefore, ADRP proteins may be produced synthetically or recombinantly by methods well known to those skilled in the art. Recombinant expression of ADRP, or fragment, derivative or analog thereof, generally comprises construction of an expression vector containing an ADRP polynucleotide that encodes the protein. The vector for the production of the protein molecule may be produced by recombinant DNA technology using techniques well known in the art. The expression vector is transferred to a host cell by conventional techniques and the transfected or transformed cells are then cultured by conventional techniques to produce the ADRP polypeptide. Alternatively, direct delivery of DNA to the cells in vivo may be used.

A variety of host-expression vector systems may be utilized. Such host-expression systems represent vehicles by which the coding sequences of interest may be produced and subsequently purified, but also represent cells which may, when transformed or transfected with the appropriate nucleotide coding sequences, express an ADRP polypeptide in situ. These include but are not limited to microorganisms such as bacteria transformed with recombinant bacteriophage DNA, plasmid DNA or cosmid DNA expression vectors containing ADRP coding sequences; yeast transformed with recombinant yeast expression vectors; insect cell systems infected with recombinant virus expression vectors (e.g., baculovirus); plant cell systems infected with recombinant virus expression vectors (e.g., cauliflower mosaic virus, CaMV; tobacco mosaic virus, TMV) or transformed with recombinant plasmid expression vectors (e.g., Ti plasmid); or mammalian cell systems (e.g., COS, CHO, BHK, VERY, Hela, MPCK, 293, 3T3 and W138 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In mammalian host cells, a number of viral-based expression systems may be utilized. In cases where an adenovirus is used as an expression vector, the coding sequence may be ligated to an adenovirus transcription/translation control complex, e.g., the late promoter and tripartite leader sequence. This chimeric gene may then be inserted in the adenovirus genome by in vitro or in vivo recombination.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the ADRP protein. Different host cells have characteristic and specific mechanisms for the post-translational processing and modification of proteins and gene products. Appropriate cell lines or host systems can be chosen to ensure the correct modification and processing of the ADRP protein expressed. To this end, eukaryotic host cells which possess the cellular machinery for proper processing of the primary transcript, glycosylation, and phosphorylation of the gene product are preferred. In particular, breast cancer cell lines such as, for example, BT483, Hs578T, HTB2, BT20 and T47D, and normal mammary gland cell line such as, for example, CRL7030 and Hs578Bst are preferred. For long-term, high-yield production of recombinant proteins, stable expression is preferred.

Once the ADRP polypeptide has been produced by an animal, chemically synthesized, or recombinantly expressed, it may be purified by any method known in the art, for example, by chromatography, centrifugation, differential solubility, or by any other standard technique for the purification of proteins. Serrero et al., 2000, Biochim. Biophys. Acta 1488: 245-254, which is herein incorporated by reference in its entirety, provides an example for a method among many for purifying recombinant ADRP expressed in bacteria.

Human ADRP proteins are preferred because they are not expected to be antigenic. However, xenogenic ADRP proteins or mimetic may also be used because even if they may have mild antigenicity in the patient, methods well-known to those in the art can be used to eliminate or minimize any side effects or ineffectiveness caused by such antigenicity. For example, coupling the protein with polyethylene glycol (PEG) is known to reduce antigenicity and increase serum half-life of foreign proteins. In addition, ADRP is a stable protein since ADRP is known to be secreted in milk.

An ADRP protein, or an ADRP polynucleotide, may be delivered via a breast cell-specific antibody. It is well known that breast cancer cells express antigens that are either cancer specific or breast cancer specific. Many breast-cell specific or breast cancer cell-specific monoclonal antibodies are available. These antibodies can be used to deliver an ADRP protein or nucleic acid molecules, or small molecules in a specific fashion. See e.g. Baluna et al., 2000, Exp. Cell Res. 258: 417-424; and Francisco et al., 1997, J. Biol. Chem. 272:24165-24169

In a specific embodiment, recombinant constructs comprising an ADRP polynucleotide for over-expression is administered to treat, inhibit and/or prevent a breast cancer, by way of gene therapy. Gene therapy refers to a therapy performed by the administration to a subject of an expressed or expressible nucleic acid molecule, preferably in the form of a recombinant construct wherein the to-be-expressed gene is under the control of one or more suitable regulatory elements.

For general reviews of the methods of gene therapy, see for example Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217). Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In general, an ADRP polynucleotide can be inserted into vectors and used as gene therapy vectors. In order to express a desired ADRP polypeptide, an appropriate expression vector should be used. An expression vector is a vector which contains the necessary elements for the transcription and translation of the inserted coding sequence. Methods which are well known to those skilled in the art may be used to construct expression vectors containing sequences encoding an ADRP polypeptide and appropriate transcriptional and translational control elements.

The “regulatory elements” or “control sequences” present in an expression vector are those non-translated regions of the vector, such as enhancers, promoters, 5′ and 3′ untranslated regions that interact with host cellular proteins to carry out transcription and translation. Such elements may vary in their strength and specificity. Depending on the vector system and host utilized, any number of suitable transcription and translation elements, including constitutive and inducible promoters, may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan et al., 1984, Proc. Natl. Acad. Sci. 81:3655-3659). In addition, transcription enhancers, such as the Rous sarcoma virus (RSV) enhancer, may be used to increase expression in mammalian host cells.

In particular, the ADRP polynucleotide has promoters operably linked to the coding region, the promoter being inducible or constitutive, and, optionally, tissue-specific. Many suitable promoters are available and are generally known to those skilled in the art

A breast cell specific promoter is preferred for the present invention Suitable breast cell-specific promoters include the ADRP promoters, whey acidic protein promoter (Li et al., 1998, Oncogene 16:997-1007), beta casein promoter (Oh et al., 1999, Transgenic Res 8:307-11), Lactalbumin promoter and Beta-lactoglobulin promoter (Brandt et al., 2000, Oncogene 19:2129-37.

Gene therapy vectors can be delivered to a subject by, for example, intravenous injection, local administration (see U.S. Pat. No. 5,328,470) or by stereotactic injection (see e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods well known in the art, e.g., by constructing them as part of an appropriate nucleic acid expression vector and administering it so that they become intracellular, e.g., by infection using defective or attenuated retroviral or other viral vectors (see e.g. U.S. Pat. No. 4,980,286), or by direct injection of naked DNA, or by use of microparticle bombardment (e.g., a gene gun), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432).

In this regard, oncogenic receptor overexpressed in breast cancer such as erbB2 may preferably be used. Normal mammary epithelial cell receptors, such as prolactin receptor (Chughtai et al., 2002, J. Biol. Chem. 277:31107-14) may also be used.

In another embodiment, nucleic acid-ligand complexes can be formed in which the ligand comprises a fusogenic viral peptide to disrupt endosomes, allowing the nucleic acid to avoid lysosomal degradation.

In yet another embodiment, the nucleic acid can be targeted in vivo for cell-specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO 92/20316 dated Nov. 26, 1992 (Findeis et al.); WO 93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, viral vectors that contain an ADRP polynucleotide are used. For example, a retroviral vector can be used (see Miller et al., 1993, Meth. Enzymol. 217:581-599). These retroviral vectors have been modified to delete retroviral sequences not necessary for packaging of the viral genome and integration into host cell DNA. The ADRP polynucleotide to be used in gene therapy is cloned into one or more vectors, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruse vectors are also often used in gene therapy. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10, demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146).

Additional viral vectors useful for delivering the polynucleotides encoding an ADRP polypeptide by gene transfer include those derived from the pox family of viruses, such as vaccinia virus and avian poxvirus. By way of example, vaccinia virus recombinants can be constructed as follows. The ADRP polynucleotide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the ADRP polypeptide into the viral genome. The resulting TK-recombinant can be selected by culturing the cells in the presence of 5-bromodeoxyuridine and picking viral plaques resistant thereto.

A vaccinia-based infection/transfection system can be conveniently used to provide for inducible, transient expression or coexpression of an ADRP polynucleotide. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the ADRP polynucleotide, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, 1990, Proc. Natl. Acad. Sci. USA, 87:6743-6747; Fuerst et al., 1986, Proc. Natl. Acad. Sci. USA, 83:8122-8126.

Alternatively, avipoxviruses, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576. Moreover, molecular conjugate vectors, such as the adenovirus chimeric vectors described in Michael et al., 1993, J. Biol. Chem. 268:6866-6869 and Wagner et al., 1992, Proc. Natl. Acad. Sci. USA 89:6099-6103, can also be used for gene delivery.

Another approach to gene therapy involves transferring a gene to cells in tissue culture by such methods as electroporation, lipofection, calcium phosphate mediated transfection. Usually, the method of transfer includes the transfer of a selectable marker to the cells. The cells are then placed under selection to isolate those cells that have taken up and are expressing the transferred gene. Those cells are then delivered to a patient.

In this embodiment, an ADRP polynucleotide is introduced into a cell to produce a recombinant cell which is then administered in vivo. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T-lymphocytes, B-lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc. In a preferred embodiment, the cell used is a breast cell autologous to the patient.

In an embodiment in which recombinant cells are used in gene therapy, an ADRP polynucleotide is introduced into the cells such that it is expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention.

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

The present invention also provides a method for screening for an agent for treating or preventing breast cancer. The method comprises generally 1) providing a cell which comprises an ADRP promoter operably linked to a reporter gene, 2) contacting a test agent with the cell, 3) determining expression level of said reporter gene in the presence of the test agent, 4) determining expressing level of said reporter gene in the absence of the test agent, and 5) selecting test agents that increases ADRP promoter activity. In a preferred embodiment, the luciferase gene may be used as a reporter gene. The mouse and human ADRP promoters have been cloned by the laboratory of the present inventor, see Eisinger and Serrero, 1993, Structure of the gene encoding mouse adipose differentiation-related protein (ADRP). Genomics. 1993 16(3):638-44. The Genbank id for the mouse ADRP promoter is gi:191730, and the Genbank id for the human promoter is gi: 16416231.

In another embodiment, the screening method of the present invention comprises 1) providing a cell which expresses ADRP protein, 2) admixing a test agent to the cell, 3) measuring an activity of ADRP in the presence of the test agent, 4) measuring an activity of ADRP in the absence of the test agent, and 5) selecting test agents that increases ADRP activity.

An ADRP activity suitable for the above screening method is lipid uptake. It has been shown that ADRP facilitates fatty acid uptake in COS cells transfected with ADRP cDNA, and that uptake of long chain fatty acids is significantly stimulated in a time-dependent fashion in ADRP-expressing COS-7 cells compared with empty vector-transfected control cells (Gao and Serrero, 1999, J. Biol. Chem. 24:16825-16830). Accordingly, oleic acid uptake velocity of ADRP-expressing COS-7 cells, or other suitable cell lines expressing ADRP may be measured in the presence or absence of a test agent, and an increase in the uptake velocity in the presence of a test agent indicates that this agent enhances ADRP activity. In a preferred embodiment, fluorescent or radiolabeled lipid may be used to facilitate the measurement of uptake velocity. Specifically, the uptake of radiolabeled fatty acid may be measured according to the method described in Example 6.

In a preferred embodiment, the present invention provides for methods using computer aided rational drug design to identify or screen for agents for breast cancer treatment.

ADRP has been expressed in recombinant form (Serrero et al, 2000, Biochim. Biophys. Acta. 1488: 245-254) and can be purified while maintaining its biological activity. Recombinant ADRP can be used for X-ray crystallography to determine 3-D structure of the protein. Combination of deletion can determine the smallest ADRP derivatives that maintains biological activity (fatty acid uptake can be used for that purpose). The smallest ADRP derivative can also be used for 3D structure determination to design molecules that bind to the active site. This information can be used for Computer aided rational drug design (docking of large chemical banks) to identify small molecules that dock to ADRP.

Once available, 3D structures of the ADRP domains will be used for database searching. Many chemical databases are available commercially. ADRP structure will be subject to molecular dynamics (MD) simulations in solution to obtain equilibrated structures for the database search. These calculations will be performed with the program CHARMM (Brooks et al., 1983, CHARMM: A Program for Macromolecular Energy, Minimization, and Dynamics Calculations. J. Comput. Chem 4: 187-217). Database searching will be carried out using a suitable program including the program DOCK 4.0.1(17) with flexible ligands based on the anchored search method 9 (Leach & Kuntz, 1992, Conformational analysis of flexible ligands in macromolecular receptor sites. J. Comput. Chem 13, 730-48).

Database searching may be performed using well-established protocols. Electrostatic charges and atom types for the proteins will be added via SYBYL. Sphere sets will be calculated with the DOCK associated program SPHGEN based on the solvent accessible surfaces of the ADRP domains. The final sphere set will be generated based on all spheres within 4.0 Å of residues of interest. Each compound in the database will initially be screened for a minimum (e.g. 10 nonhydrogen atoms) and maximum size (e.g. 40 nonhydrogen atoms) and for the number of rotatable bonds (e.g. less then 15). Selected compounds will next be built into the binding site by placing “anchor fragments” (i.e. individual rigid segments with five or more heavy atoms, such as aromatic rings) in 200 different orientations that maximize overlap with the sphere set. The remainder of each molecule will then be added to the anchor fragment in a gradual build-up procedure that includes determination of the lowest energy conformation of the added portion of the molecule. Interaction energies will be approximated by the sum of electrostatic and Van der Waals components as calculated by the GRID method (Meng et al., 1994, Evaluating docked complexes with HINT exponential functional and empirical atomic hydrophobicities. J. Comput. Aided Mol. Design 8: 299-306) implemented in DOCK. The orientation with the most favorable total interaction energy will be determined for each compound with the top 10,000 compounds selected for further study. Additional screening of the selected 10,000 compounds will be performed by more a rigorous docking method (“Method 2”) that includes simultaneous energy minimization of the anchor fragment during the iterative build-up procedure and will be performed on about 5 modeled structures. The final score for ranking of each compound will be the average of the total interaction energy from the 5 Method-2 dock runs. The use of the average scores for the 5 modeled structures allows for the protein flexibility to taken into account. The top 1000 compounds from the Method 2 search will be selected for further analysis.

The final step of the selection process is designed to maximize the chemical diversity of compounds for testing. To achieve this, clustering approaches based on chemical similarity will be applied (Butina, 1999, Unsupervised database Clustering on Daylight's Fingerprint and Tanimoto Similarity: A Fast and Automated Way to Cluster Small and Large Data Sets. J. Chem. Inf. Comput. Sci. 39, 747-750). Chemical similarity is based on chemical “fingerprints.” The fingerprint of each compound is based on the types of atoms in the compound and the connectivity between those atoms: atoms bonded to each other, atoms bonded to one of the atoms in the first bonded pair, and so on. Compounds with similar fingerprints will be clustered using the Tanimoto Similarity Index. Typically, approximately 100 clusters of compounds are obtained. Compounds in each cluster will then be analyzed for stability, potential toxicity, solubility and synthetic feasibility with one or two compounds selected for biological assay, yielding a total of approximately 200 compounds for biological assay. Selected compounds will then be purchased from the appropriate vendor or synthesized or produced.

In additional embodiments, the present invention concerns formulation of an ADRP polynucleotide, polypeptide, or agonist disclosed herein in pharmaceutically-acceptable carriers for administration to a patient, either alone, or in combination with one or more other modalities of therapy.

It will be understood that, if desired, a composition as disclosed herein may be administered in combination with other agents as well, given that the additional agents do not cause a significant adverse effect upon contact with the target cells or host tissues.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration. Carriers for use within such pharmaceutical compositions are biocompatible, and may also be biodegradable.

The pharmaceutical compositions described herein are administered to a patient, who may or may not be afflicted with breast cancer. Pharmaceutical compositions and vaccines may be administered either prior to or following surgical removal of primary tumors and/or treatment such as administration of radiotherapy or conventional chemotherapeutic drugs. As discussed above, administration of the pharmaceutical compositions may be by any suitable method, including administration by intravenous, intraperitoneal, intramuscular, subcutaneous, intranasal, intradermal, anal, vaginal, topical and oral routes.

Routes and frequency of administration of the therapeutic compositions described herein, as well as dosage, will vary from individual to individual, and may be readily established using standard techniques. In general, an appropriate dosage and treatment regimen provides the active compound(s) in an amount sufficient to provide therapeutic and/or prophylactic benefit.

### EXAMPLES

### Example 1

### Growth of Breast Cancer Cells Over-Expressing ADRP is Inhibited Even in the Presence of Growth Stimulating Factors and Growth Hormones

A human estrogen receptor positive (ER⁺) mammary epithelial cancer cell line, MCF-7, is routinely used for the study of ER⁺ human breast cancer. These cells proliferate under the stimulation of estradiol, and are growth inhibited by tamoxifen and other anti-estrogen therapy. They also can grow in anchorage independent conditions (a hallmark of tumorignic cells) and tumors when injected into nude mice after estrogen supplementation.

The vector used to over-express ADRP in MCF-7 cells is the ADRP-pcDNA3 vector described in Gao and Serrero, 1999. The G418 resistance gene was used as selectable marker. MCF-7 cells were transfected with the ADRP-pcDNA3 plasmid, using the lipofectamine method. Specifically, MCF-7 cells (WT) were plated onto 60-mm dish at the concentration of 2×10⁵ cell per plate in 5% fetal bovine serum (FBS) in DMEM/F12 (1:1 mixture of Dulbecco's modified Eagle's medium and Ham' nutrient F12 medium) overnight before transfection. Cells were washed once with serum-free DMEM/F12 (without antibiotic). The lipofectamine method was used for the transfection. Specifically, 2 μg mouse ADRP pcDNA3 construct was used. Transfection was carried on according to manufacturer's recommendations, with 15 μl of lipofectamine and 5 hours of incubation. After transfection, the medium was replaced with 5% FBS DMEM/F12 and the cells were incubated overnight. G418 at the concentration of 800 μg/ml was added the day after for selection. Cells were selected with this medium for 3 weeks. Six clones were isolated and subjected to RT-PCR to determine the existence of the transfection construct.

As control, MCF-7 cells were transfected by the same method with empty pcDNA3 vector. Cells transfected with empty vector (EV-MCF-7 cells) were used as control in all our experiments.

Immunohistochemistry (IHC) staining indicated that ADRP was expressed on the cell surface of the lipid droplets (Gao and Serrero, 1999). By immunofluorescence staining of fixed cells with anti-ADRP antibody followed by FITC-conjugated anti-rabbit secondary antibody, ADRP in transfected COS-7 cells was localized primarily at the cell periphery. Additional staining was also found associated with the nucleus of transfected cells as well as in control cells although with a lesser degree. These data suggest that ADRP is preferentially found associated with the plasma membrane in the transfected cells.

Clones that stably expressed ADRP cDNA were selected and frozen for future study. We analyzed the proliferation of these cells. This was prompted by the fact that to our surprise the ADRP transfected cells grew very slowly particularly when compared to cells transfected with the empty vectors.

For proliferation assay, the cells were seeded at a density of 5×10⁴ cells in phenol red-free α-MEM (PFME) supplemented with 5% of charcoal extracted fetal bovine serum (CHX-FBS) in 6-well culture plates (Costar). Cells were treated with 1 nM 17β-estradiol alone. Control cells were treated with vehicle alone (0.01% DMSO). Cell numbers were counted by hemacytometer every two days. Medium was changed at day 4. Each time point was performed in triplicate.

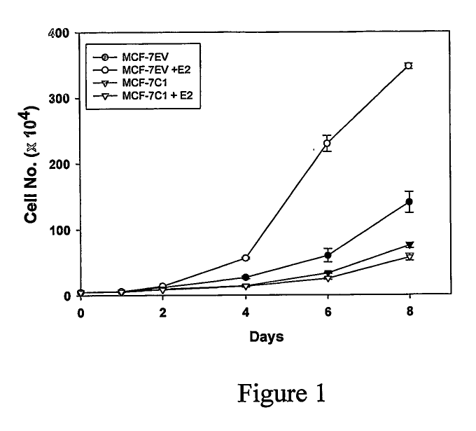

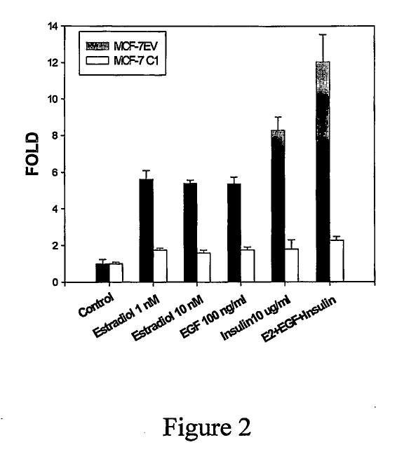

As shown in FIGS. 1 and 2, cells over-expressing ADRP grow very slowly both in serum containing medium and also when the medium was supplemented with hormones and growth factors known to stimulate the proliferation of mammary epithelial cells.

Specifically, the medium contained estradiol (E2 at 10⁻⁹ M), insulin (10 microgramsg/ml) and epidermal growth factor (EGF at 25 ng/ml). We showed that these three factors stimulated the proliferation of EV MCF-7 cells (control), whereas they had no effect on the ADRP overexpressing cells.

### Example 2

### Differentiation of Breast Cancer Cells Expressing ADRP is Stimulated

We then explored whether growth inhibition of cells overexpressing ADRP was accompanied by stimulated differentiation of the cells. In order to investigate this possibility, we measured the expression of markers of differentiation such as beta casein in the ADRP expressing cells when compared to EV cells. Beta casein is routinely used as a marker of mammary epithelial cell differentiation.

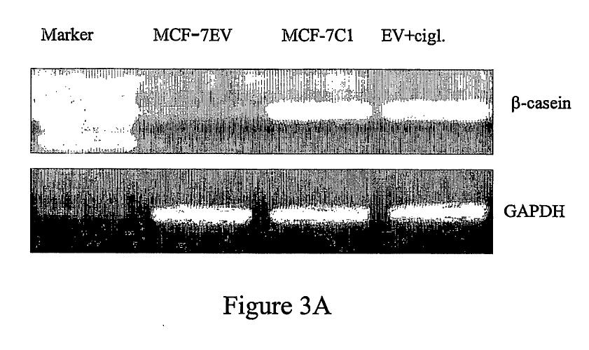

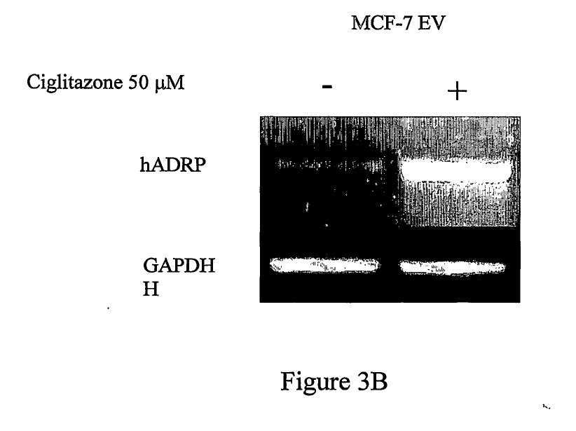

We show by RT-PCR that the ADRP overexpressing cells express high level of casein mRNA. In contrast, the control cells had no detectable level of the gene product (see FIG. 3A). In FIG. 3A, GAPDH (Glyceraldehyde-3-phosphate dehydrogenase) loading was used as an internal control for equal sample mRNA expression. GAPDH expression does not change between the two cell types, and is routinely used as an internal control.

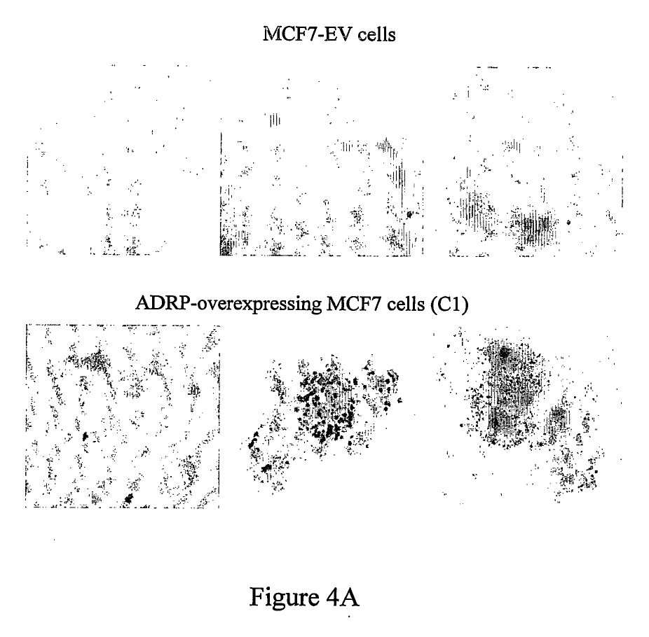

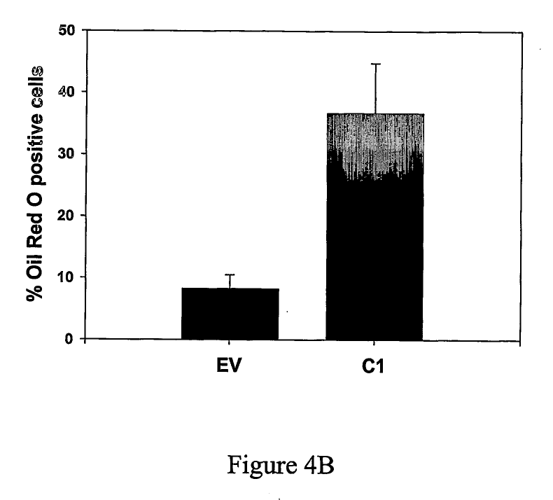

This is also supported by the fact that ADRP overexpressing cells accumulated lipid droplets at a higher level (higher number of droplets per cells and higher number of cells having droplets than the empty vector control MCF-7 cells as shown in FIG. 4A and FIG. 4B. A 5-fold increase in lipid droplets accumulation was observed (FIG. 4B). Specifically, cells were plated at the concentration of 2×10⁵ per 35-mm dish in 5% FBS DMEM/F12. After 48 h of incubation, cells were washed once with PBS and fixed in 4% formalin for 30 min. Oil Red O working solution was added to cover all the cells, and the cells were incubated at 37° C. for 1 h. The Working Solution was prepared by mixing 6 ml 0.5% Oil Red O in isopropanol with 4 ml H₂O, and centrifuging at 4000 g for 10 min to remove sediments. The plates were washed with tap water twice. Cells were counterstained by Hematoxylin QS (Vector Laboratories, California) for 45 sec, and washed with tap water.

### Example 3

### Agents that Stimulate Cell Differentiation or Inhibit Proliferation also Stimulate ADRP Expression

In order to confirm that ADRP expression is linked to the differentiation program we proved that agents that have been described as inhibiting proliferation and increasing differentiation of mammary epithelial cells do also stimulate ADRP expression. Ciglitazone is known to bind and activate the nuclear receptor PPAR gamma, which has been shown in MCF-7 to stimulate expression of beta casein (Mueller et al., 1998). We show that treatment with ciglitazone 50 μM stimulates casein expression (differentiation marker) as well ADRP expression in control MCF-7 cells (FIGS. 3A and 3B).

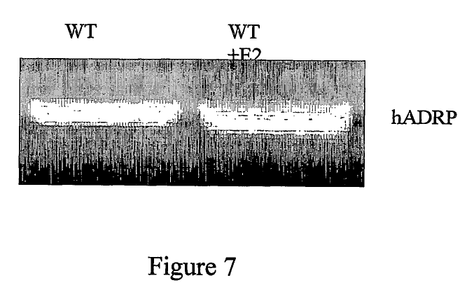

It is known that estradiol plays a dual role in mammary epithelial cells as a stimulator of proliferation and also as a differentiation factor at different times of the developmental stages of mammary gland development (Kumar et al, 2000). We also showed that estradiol stimulates ADRP expression in MCF-7 cells (FIG. 7). This indicates that ADRP expression in mammary epithelial cells is under the control of physiological regulators of mammary gland development, and that high ADRP expression is associated with expression of differentiated phenotype.

### Example 4

### Overexpression of ADRP Alone is Sufficient to Induce Differentiation and Inhibit Proliferation Including Anchorage-Independent Growth, and Stimulate Apoptosis

The overexpression studies show that ADRP expression alone is sufficient to stimulate the cells into becoming growth inhibited and differentiated.

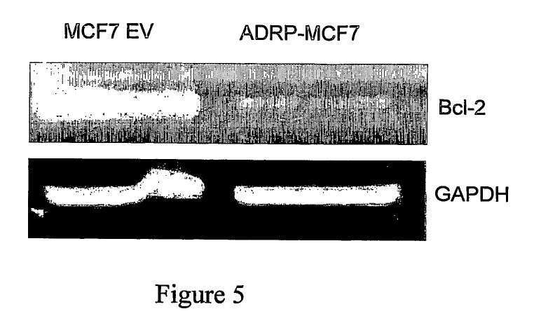

In addition, we showed that expression of ADRP stimulates the cells into undergoing apoptosis. This is shown by down-regulation of bcl-2 in ADRP MCF-7 cells when compared to control MCF-7 cells. FIG. 5 shows that Bcl-2 expression is dramatically decreased in ADRP-overexpressing cells when compared to empty vector control cells. Bcl-2 is a marker that is upregulated in cancer cells, and its upregulation is associated with decreased apoptosis and increased survival of cancer cells (Murphy et al., 1999, bcl-2 Expression Delays Mammary Tumor Development in Dimethylbenz(a)anthracene-treated Transgenic Mice, Oncogene 18, 6597-6604.). This indicates that when ADRP is expressed in the cells, they will become more susceptible to apoptosis and to apoptosis-inducing stimuli such as cytotoxic agents, including chemotherapeutic agents such as doxorubicin, and taxol and radiation therapy. In other words, ADRP overexpression sensitizes the cells to apoptotic stimuli.

Increased of percentage of cells in apoptosis and in G1 phase is shown by FACS analysis of ADRP-MCF-7 cells and EV-MCF-7 cells.

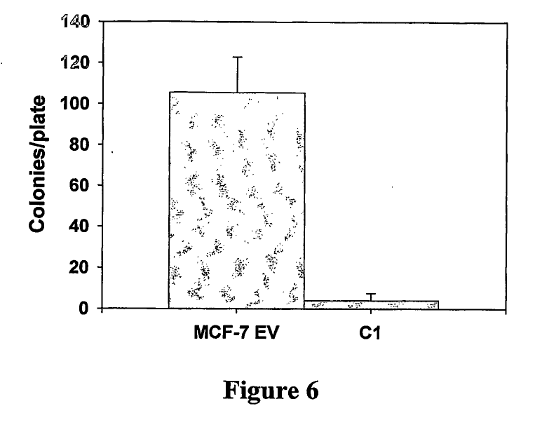

Overexpression of ADRP in MCF-7 cells leads to a more than 98% inhibition of anchorage independent growth of the breast cancer cells MCF-7 cells. It is known that anchorage independent growth of cells in soft agar is a measure of tumorigenesis of cancer cells. Normal cells cannot grow in soft agar whereas tumorigenic cells can. Cells growing in soft agar form colonies that can be counted. The data of FIG. 6 indicate that overexpression of ADRP suppresses the tumorigenic properties (tumorigenesis) of the MCF-7 cells (FIG. 6).

Anchorage independent growth was determined by soft agar colony forming assay. MCF-7 cells were trypsinized by trypsin-EDTA solution for 5 minutes. 10,000 cells in 1 ml of 0.33% agar in tissue culture medium containing 10% FBS in 35-mm plates were placed over a bottom layer of 1.5 ml of 0.6% agar in DMEM/F12+10% FBS. The cells were allowed to grow for 20 days with 200 μl of DMEM/F12 refeeding every 3 days. Colonies were stained with 0.005% crystal violet for an hour. Experiments were carried out in quadruplicate.

In summary, overexpression of ADRP in human breast cancer cells is both necessary and sufficient to stimulate breast cancer cell differentiation, inhibit proliferation, and tumorigenesis and to increase apoptosis, which in turn results in reducing cancer cell survival. ADRP, therefore, is an excellent factor as anti-cancer agent. Furthermore, stimulating endogenous ADRP expression or by otherwise activating ADRP function in vivo can serve as breast cancer chemoprevention because high ADRP level maintain cells in the differentiated state. This will reduce the possibility of these cells being stimulated to proliferate, as it is the case in tumorigenesis.

### Example 5

### Measuring Uptake of Radio-Labeled Fatty Acids

1) Preparation of Albumin-Bound Fatty Acids

9,10-[³H] oleic acid (1.2 μCi/mmol) (Dupont-NEN, Wilmington, Del.) and unlabeled oleic acid Na salts are dissolved in 10 ml water at 40° C. to give a concentration of about 320 μM (48 μCi/ml). When the solution is completely clear in around 10 min, fatty acid-free BSA (Sigma, St Louis, Mo.) from a concentrated stock solution (20%) was added with gentle mixing to obtain a final concentration of 80 μM of BSA and an oleic acid: BSA molar ratio of 4.0. For the assays, an aliquot of the stock solution of FA-BSA (320 μM was 1:8 diluted with PBS containing or not additional fatty acid-free BSA to obtain the final desired concentrations of oleic acid (40 μM) and of fatty acid-free BSA (10 μM, 13 μM, 20 μM, 40 μM, and 80 μM) and oleic acid/BSA molar ratios of 4.0, 3.0, 2.0, 1.0, and 0.5, respectively. The final concentration of fatty acids in the assay was 20 μM [³H] oleic acid (3 μCi/ml) with various concentrations of fatty acid-free BSA. The unbound oleic acid concentration in the presence of BSA was determined from the oleate:BSA molar ratio according to the calculation of Abumrad et al (13), using the association constants of Spector et al (25) and the model of stepwise equilibrium developed by Klotz et al (26). 9,10-[³H] palmitic acid (43 μCi/nmol), 5,6,8,11,12,14,15-[³H] arachidonic acid (222 μCi/nmol) and 1-[¹⁴C]octanoic acid (55 μCi/nmol) (Dupont-NEN, Wilmington, Del.) were diluted to 40 μM with a fatty acid-free BSA concentration of 10 μM. The final working concentration in the tubes was 20 μM of [³H]-palmitic acid (3.1 μCi/ml), [³H]-arachidonic acid (1.3 μCi/ml) and [¹⁴C] octanoic acid (12 nCi/ml), respectively, with 5 μM fatty acid-free BSA.

2) Radiolabeled Fatty Acid Uptake by Transfected Cos 7 Cells.

Transfected Cos-7 cells were resuspended in PBS (5×10⁵ cells/ml) in 15 ml polypropylene centrifuge tubes. 200 μl aliquots of cell suspension (10⁵ cells) were placed in 5 ml polypropylene centrifuge tubes. Cell suspensions in PBS were pre-incubated for 5 min at 37° C. in a shaking water bath. An equal volume of 2-fold concentrated stock fatty acid/BSA solutions was added to each tube to perform fatty acid uptake. At specific intervals, uptake was stopped by adding 5 ml of ice-cold PBS containing 0.1% BSA and 200 μM phloretin (wash solution) into each assay tube. Then the solutions and cell suspensions were filtered through GF/C filters (Whatman, Maidstone, England) which had been presaturated with 15 ml of 0.1% BSA in PBS. Cells retained by filters were rapidly washed 3 times with 5 ml of cold wash solution. Filters were soaked overnight in 10 ml of scintillation cocktail prior to counting with a liquid scintillation counter. Non-specific fatty acid adsorbed to filters lacking cells was routinely measured and subtracted from experimental values. Background radioactivity representing isotope trapped extracellularly and bound nonspecifically by the cells was measured from zero time incubation determined by adding stop solution to the cells before adding radiolabeled fatty acid-BSA complex.

Fatty acid uptake data were normalized with the transfection efficiency that had been determined by counting the number of fluorescent cells expressing GFP compared to the total cell number. Since non-transfected cells could also uptake fatty acid, it was necessary to remove their contribution to the total FA uptake in order to determine the contribution due to the expression of ADRP in the cells. To achieve this, the uptake of FA by non-transfected cells was determined by multiplying the value of total FA uptake in non-transfected control cells by the percentage of non-transfected cells (100%-transfection efficiency). Non-transfected control cells were treated similarly to transfected cells but without adding plasmid DNA of vectors used for transfection. The value for the remaining FA uptake contributed by the transfected cells was obtained by subtracting the nontransfected cells uptake from the total uptake measured experimentally. Then the remaining FA uptake of transfected cells was normalized to the transfection efficiency. The final amount of uptake was expressed as pmol per 10⁵ transfected cells.

3) Binding of ADRP to NDP-Stearate and Cis-Parinaric Acid (Fluorescent Fatty Acids):

The binding of recombinant ADRP protein to fluorescent fatty acid derivatives was determined as described in Stolowich, et al., 1997, Biochemistry, 36:1719-1729, and Schroeder et al., 1995, Biochemistry, 34:11919-11927. with the following modifications: a 2 ml sample of 0.1 μM rADRP was titrated with small increments of fatty acid (0.1 to 1 μl) dissolved in EtOH. Concentration of the fatty acid stock solution was 460 □M. Each sample and the blank (without ADRP) were thoroughly mixed and allowed to equilibrate for 1-2 minutes prior to initiating the measurement in order to achieve maximal fluorescence signal. All measurements were performed at 25° C. Total sample absorbance at the wavelength of excitation was less than 0.15. Steady state fluorescence spectra were measured on a PC1 photon counting spectrofluorometer (ISS Instr., Champaign, Ill.) using 1 cm quartz cuvette. Sample temperature was kept at 25° C. (±1° C.) in a thermostated cell holder. Excitation and emission bandwidths were 4 and 8 nm, respectively.

Binding of sterol or cholesterol: NBD-cholesterol Binding to ADRP-Recombinant mouse ADRP used in this experiment was purified according to Serrero et al, Biochim. Biophys. Acta. 1488: 245-254. The affinity of ADRP for NBD-cholesterol (NBD-chol) was determined using a steady state photon counting fluorimeter (see below) according to a modification of a previously described procedure. Briefly, ADRP was added to a 2-ml sample of phosphate buffer (10 mM, pH 7.4) to a final concentration of 11.1 nM. Small increments (0.5-2.0 μl) of NBD-chol (0.14 μM in dimethylformamide) were then added and each sample and blank (without ADRP) were mixed and equilibrated at 25° C. for 2-4 min for stable measurement of fluorescence. NBD-chol was excited at 465 nm (8 nm slits) and fluorescence emission spectra were recorded from 500 to 600 nm (16 nm slits). The NBD-chol fluorescence emission was integrated after each addition of the ligand and corrected for the blank and background. This allowed binding isotherm construction and fitting using a simple, single binding site model as described.

### Example 6

### Inhibition of ADRP in Breast Cancer Cells by ADRP siRNA Blocks Breast Cancer Cells Differentiation

Data in this example show that cells whose ADRP expression has been inhibited via siRNA transfection will not differentiate, as determined by measuring the expression of beta casein, a differentiation marker. This was the case even when the cells were treated with ciglitazone, which normally induces differentiation. On the other hand, PPAR-γ activity was not blocked. These data indicate that the key regulator for promoting breast cancer cell differentiation and for inhibiting breast cancer cells growth is ADRP, and that the target of ciglitazone action with regard to differentiation stimulation is ADRP, not PPAR-γ.

A. Methods:

A specific siRNA against human ADRP mRNA (hADRP-siRNA) was designed using the sequence between +225 to +244 of human ADRP as a target. A control siRNA was designed using a portion of the green fluorescence protein gene. Twenty-one-oligonucleotide RNA for hADRP (rArUrU rGrCrA rGrUrU rGrCrC rArArU rArCrC rUTT and rArGrG rUrArU rUrGrG rCrArA rCrUrG rCrArA rUTT) with 3′dTdT overhangs, and for GFP (rGrGrC rUrArC rGrUrC rCrArG rGrArG rCrGrC rArCrC and rUrGrC rGrCrU rCrCrU rGrGrA rCrGrU rArGrC rCrUrU) were synthesized (Integrated DNA Technologies, Inc., Coralville, Iowa). Transfection of siRNA duplex was performed in MCF-7 cells using LipofectAMINE 2000 reagents as described by the manufacturer (Invitrogen, Carlsbad, Calif.). MCF-7 EV grown to 50% confluency in 6-well plates were transfected with 100 pmole of siRNA per well in serum free-DMEM/F12. After 5 h, the medium was replaced with DMEM/F12 containing 5% FBS. Control MCF-7 cells were treated with GFP siRNA. The cells were then treated with 50 μM (50 micromolars) ciglitazone. After 48 h of incubation, RNA samples were collected by Trizol method to measure human ADRP expression and beta casein expression by RT-PCR as described above.

Measurement of Beta casein expression as marker of breast cancer cell differentiation was by RT-PCR as described above.

Measurement of PPAR gamma activity was by determining the activity of a reporter gene containing PPAR gamma responsive element (PPRE) ligated to a luciferase reporter gene. When PPRE is activated by PPAR gamma, there is an increase in luciferase activity. MCF-7 cells treated with human ADRP-siRNA and ciglitazone were transfected with 1 micrograms of PPRE reporter gene construct. The cells were co-transfected with 0.25 micrograms of CMV-beta galactosidase plasmid DNA as control to normalize the transfection efficiency.

B. Results:

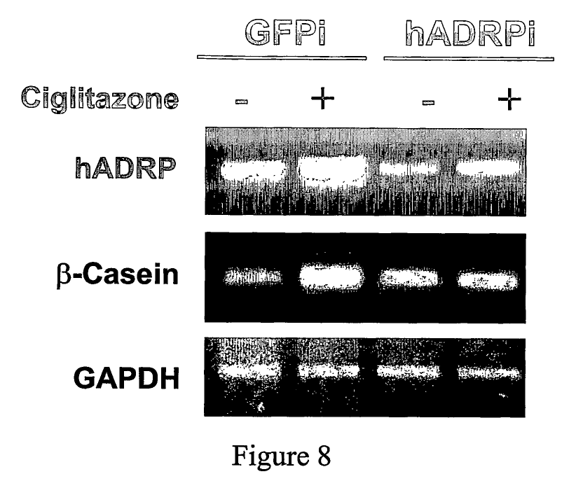

Inhibition of ADRP by siRNA prevents cell differentiation. As shown in FIG. 8, human ADRP siRNA showed strong inhibition of ADRP mRNA expression and prevented ciglitazone-induce β-casein expression (FIG. 1). In contrast, ADRP expression and β-casein expression were unaffected in GFP-siRNA transfected MCF-7 cells treated with ciglitazone. These results indicate that hADRP siRNA silenced an essential component for cell differentiation in MCF-7 cells.

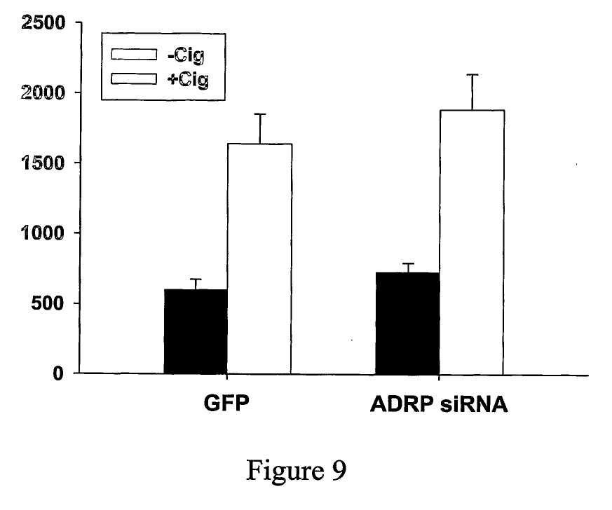

ADRP siRNA Transfection Did Not Prevent the Increase in PPRE Activity in MCF-7 Cells Treated With Ciglitazone. It is known that ciglitazone is a PPAR gamma ligand and therefore an activator of PPAR gamma activity. This activity can be measured by determining the increase of a luciferase reporter gene construct containing PPAR gamma response element (PPRE-Luc). We determined the ability of ciglitazone to activate PPAR gamma when differentiation had been blocked by inhibiting ADRP expression by siRNA. MCF-7 cells transfected with hADRP-siRNA and treated with ciglitazone were transfected with the PPRE reporter gene construct. As shown in FIG. 9, PPRE activity was the same in MCF-7 cells treated with ciglitazone and transfected with GFP-si RNA positive control) or with H-ADRP siRNA independently whether the cells were differentiated or not. This proves that ADRP, PPAR-γ, is the key regulator of breast cancer cell differentiation, and that ciglitazone stimulates differentiation by stimulating ADRP.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. All publications, patents and patent applications cited above are hereby incorporated by reference in their entirety.

What is claimed is:

1. A method for treating or preventing breast cancer in a patient, the method comprising inducing ADRP gene over-expression in a breast cell of the patient.

2. A method of claim 1, wherein the breast cell is a cancer cell.

3. A method of claim 1, wherein the method comprises increasing ADRP level or inducing endogenous ADRP gene expression of said breast cell.

4. A method of claim 3, further comprising measuring ADRP gene expression level.

5. A method of claim 4, wherein the ADRP gene expression level is measured by the level of ADRP mRNA.

6. A method of claim 4, wherein the ADRP gene expression level is measured by the level of ADRP protein.

7. A method of claim 3, wherein the endogenous ADRP gene is induced to over-express by administering an effective amount of a compound selected from the group consisting of a 1-PPARγ ligand, 9-cis retinoic acid, a long chain fatty acid, estradiol, and a 4-Cyclooxygenase inhibitor.

8. A method according to claim 7, wherein the PPARγ ligand is ciglitazone or thialozidinedione (TZD).

9. A method according to claim 7, wherein the long chain fatty acid is a non-metabolizable long chain fatty acid.

10. A method according to claim 9, wherein the long chain fatty acid is bromopalmitate.

11. A method according to claim 7, wherein the 4-Cyclooxygenase inhibitor is indomethacin or ibuprofen.

12. A method of claim 1, wherein the ADRP level of said cell is increased by delivering ADRP protein to said cell.

13. A method of claim 2, wherein the ADRP protein is delivered to said cell via a breast cell-specific antibody.

14. A method of claim 1, wherein the method comprising introducing an exogenous polynucleotide encoding ADRP into said breast cell and expressing said exogenous ADRP gene.

15. A method according to claim 14, wherein the exogenous polynucleotide encoding ADRP is operably linked to a promoter.

16. A method according to claim 15, wherein the promoter is a mammary tissue-specific promoter.

17. A method according to claim 15, wherein the mammary tissue-specific promoter is selected from the group consisting of an ADRP promoter, whey acidic protein promoter, beta casein promoter, Lactalbumin promoter and Beta-lactoglobulin promoter.

18. A method according to claim 15, wherein the promoter is a constitutive promoter.

19. A method according to claim 2, whereby the cancer cell is sensitized and becomes responsive to a cytotoxic agent.

20. A method according to claim 2, further comprising administering to the patient a cytotoxic agent for killing the cancer cell.

21. A method according to claim 20, wherein the cytotoxic agent is a compound for cancer chemotherapy or a compound for cancer radio therapy.

22. A method for screening for an agent for treating or preventing breast cancer, said method comprising
1) providing a cell which comprises an ADRP promoter operably linked to a reporter gene,
2) admixing a test agent to the cell,
3) determining expression level of said reporter gene in the presence of the test agent,
4) determining expressing level of said reporter gene in the absence of the test agent, and
5) selecting test agents that increases ADRP promoter activity.

23. A method according to claim 22, wherein the cell is selected from the group consisting of Cos-7 cells, CHO cells, human 293 cells, Hela cells and mammary epithelial cells.

24. A method according to claim 22, wherein the reporter gene is a luciferase gene.

25. A method for screening for an agent for treating or preventing breast cancer, said method comprising
1) providing a cell which expresses ADRP protein;
2) admixing a test agent to the cell,
3) measuring an activity of ADRP in the presence of the test agent,
4) measuring an activity of ADRP in the absence of the test agent, and
5) selecting test agents that increase the ADRP activity.

26. A method according to claim 25, wherein the ADRP activity is selected from the group consisting of fatty acid uptake, fatty acid binding, induction or increase of a differentiation marker of mammary epithelial cells.

27. A method of claim 26, wherein the differentiation marker is beta casein expression or accumulation of lipid droplets

28. A method of claim 26, wherein the ADRP activity is lipid uptake of ADRP-expressing cells.

29. A method of claim 25, wherein the ADRP activity is stimulation of breast cancer cell differentiation, inhibition of proliferation of human breast cancer cells, or stimulation of breast cancer cell apoptosis.

30. A method for screening for an agent for treating or preventing breast cancer, said method comprising
1) admixing a test agent with a preparation of ADRP protein,
2) measuring an activity of ADRP in the presence of the test agent,
3) measuring an activity of ADRP in the absence of the test agent, and
4) selecting test agents that increase the ADRP activity.

31. A method according to claim 30, wherein the ADRP activity is binding of the ADRP protein to a fatty acid.

32. A method according to claim 30, wherein the fatty acid is a fluorescent fatty acid derivative.

33. A method according to claim 32, where the fluorescent fatty acid derivative is 12-(N-methyl)-N-(7-nibrobenz-2-oxa-1,3-diazol-4-yl) aminooctadecanoic acid (NBD-stearate), and wherein an increase in fluorescence indicates binding of ADRP to NBD-stearate.

34. A method according to claim 30, wherein the test agent is identified via computer aided drug design.

35. A method according to claim 30, wherein the method is a high-throughput screening method.

36. A pharmaceutical composition comprising an effective amount of an ADRP polypeptide or polynucleotide or an ADRP agonist, for breast cancer treatment, and a pharmaceutically acceptable excipient.
